(19) United States
(12) Patent Application Publication
(10) Pub. No.: US 2025/0375310 A1
(43) Pub. Date: Dec. 11, 2025

(54) URINE COLLECTION DEVICES HAVING A RELATIVELY WIDE PORTION AND AN ELONGATED PORTION AND RELATED METHODS

(71) Applicant: PureWick Corporation, Covington, GA (US)

(72) Inventors: James David Hughett, SR., Conyers, GA (US); Rodrigo Fernandez, Loganville, GA (US); Melissa Young Joyner, Stone Mountain, GA (US); Wanfei Yang, Decatur, GA (US); Larry Dean Alder, Newborn, GA (US); Kuilin Lai, Watkinsville, GA (US); Nathaniel Barnes, Covington, GA (US)

(21) Appl. No.: 19/237,368

(22) Filed: Jun. 13, 2025

Related U.S. Application Data
(63) Continuation of application No. 17/758,316, filed on Jul. 1, 2022, now Pat. No. 12,350,190.
(60) Provisional application No. 62/956,770, filed on Jan. 3, 2020.

(51) Int. Cl. A61F 5/455; A61F 5/453; A61F 5/48

(52) U.S. Cl. CPC A61F 5/4556; A61F 5/453; A61F 5/485

(57) ABSTRACT

Examples relate to devices, systems, and methods for fluid collection such as urine. The urine collection device includes a fluid impermeable barrier and a fluid permeable body. The fluid impermeable barrier at least partially defines a chamber. The fluid impermeable barrier includes at least one elongated portion having a first width and distal to the aperture and a second portion having a second width greater than the first width. The second portion at least partially defines an opening in the fluid impermeable barrier, and the opening has a maximum lateral dimension greater than the first width of the elongated portion. The fluid permeable body is configured to wick fluid away from the opening into the at least one elongated portion.

### CROSS-REFERENCE TO RELATED APPLICATIONS

This application is a continuation of U.S. patent application Ser. No. 17/758,316 filed on Jul. 1, 2022, which is a U.S. Nationalization of PCT International Application No. PCT/US2020/067455 filed on 30 Dec. 2020, which claims priority to U.S. Provisional Patent Application No. 62/956,770 filed on Jan. 3, 2020, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of urine collection devices continue to seek new and improved devices, systems, and methods to collect urine.

### SUMMARY

Embodiments disclosed herein are related to urine collection devices and methods of using urine collection devices. In an embodiment, a urine collection device includes a fluid impermeable barrier and a fluid permeable body. The fluid impermeable barrier at least partially defines a chamber and includes at least one elongated portion and a second portion. The at least one elongated portion has a first width. The second portion has a second width greater than the first width, and at least partially defines an aperture distal to the at least one elongated portion and an opening having a maximum lateral dimension greater than the first width. The fluid permeable body is positioned at least partially within the chamber to extend across at least a portion of the opening and into at least a portion of the at least one elongated portion of the fluid impermeable body. The fluid permeable body is configured to wick fluid away from the opening into the at least one elongated portion.

In an embodiment, a urine collection device includes a receptacle and fluid permeable body. The receptacle is sized to receive a head of a penis therein with at least a portion of an inner surface of the receptacle contacting the head of the penis. The fluid permeable body defines at least a portion of the receptacle, and the fluid permeable support is configured to wick fluid away from the receptacle for removal from the urine collection device.

In an embodiment, a method of collecting urine is disclosed. The method includes positioning a fluid permeable body of a urine collection device at least proximate to a urethra of a user. The fluid permeable body extends at least partially across an opening defined by a fluid impermeable barrier of the urine collection device. The method also includes receiving fluids discharged from the urethra into a reservoir void of material of the urine collection device, the reservoir being at least partially defined by a portion of the fluid permeable body and a portion of the fluid impermeable barrier. The method also includes promoting urine from the reservoir through a conduit fluidly coupled to the reservoir.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a front plan view of a urine collection device, according to an embodiment.

FIG. 1B is a cross-sectional view of the urine collection device of FIG. 1A taken along line A-A.

FIG. 2A is a front plan view of a urine collection device, according to an embodiment.

FIG. 2B is a front cross-sectional view of the urine collection device of FIG. 2A taken along line B-B.

FIGS. 3A-3C are isometric views of a urine collection system, according to an embodiment.

FIG. 3D is a side view of the urine collection system of FIGS. 3A-3C.

FIGS. 4A-4B are isometric views of a urine collection device, according to an embodiment.

FIG. 5 is a block diagram of a urine collection system, according to an embodiment.

FIG. 6 is a flow diagram of a method for collecting urine, according to an embodiment.

### DETAILED DESCRIPTION

Embodiments disclosed herein are related to urine collection devices and methods of using the same. The devices and systems disclosed herein are configured to collect fluids from an individual. The fluids collected by the urine collection devices may include at least one of urine, vaginal discharge, penile discharge, reproductive fluids, blood, sweat, or other bodily fluids. Conventional urine collection devices, particularly for males, may be difficult to retain in place with a fluid permeable portion of the urine collection device proximate to or adjacent to a urethra of the user. Many embodiments described herein provide securement elements and/or a larger surface area to collect urine discharged from the urethra, while also including a wicking material that promotes flow of discharged urine to a reservoir for removal from the urine collection device.

Urine collection devices described herein may be used in urine collection systems. The urine collection systems can include a urine collection device, a fluid storage container, and a portable vacuum source. Fluid (e.g., urine or other bodily fluids) collected in the urine collection device may be removed from the urine collection device via a conduit which protrudes into an interior region of the urine collection device. For example, a first open end of the conduit may extend into the urine collection device to be in fluid communication with a reservoir therein. The second open end of the conduit may extend into the urine collection container or the portable vacuum source. The suction force may be introduced into the interior region of the urine collection device via the first open end of the conduit responsive to a suction (e.g., vacuum) force applied at the second end of the conduit. The suction force may be applied to the second open end of the conduit by the portable vacuum source either directly or indirectly.

In some embodiments, the portable vacuum source may be disposed in or on the urine collection device. In such embodiments, the conduit may extend from the urine collection device and attach to the portable vacuum source at a first point therein. An additional conduit may attach to the portable vacuum source at a second point thereon and may extend out of the urine collection device, and may attach to the fluid storage container. Accordingly, a vacuum (e.g., suction) May be drawn through urine collection device via the fluid storage container. Fluid, such as urine, may be drained from the urine collection device using the portable vacuum source.

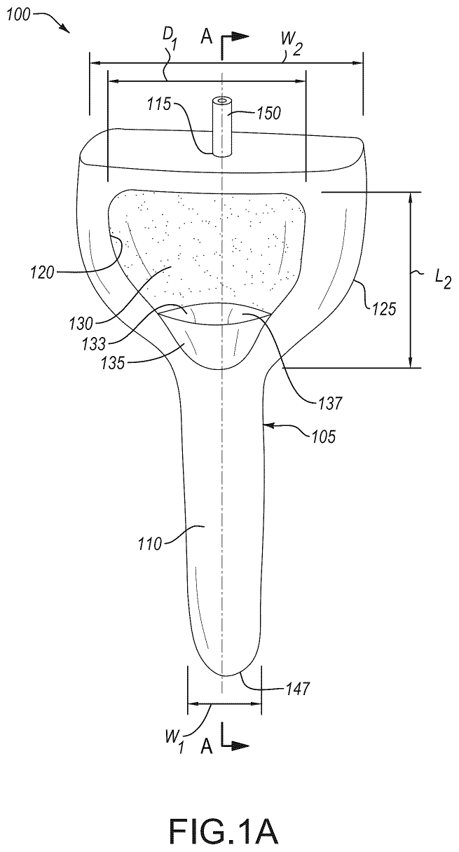

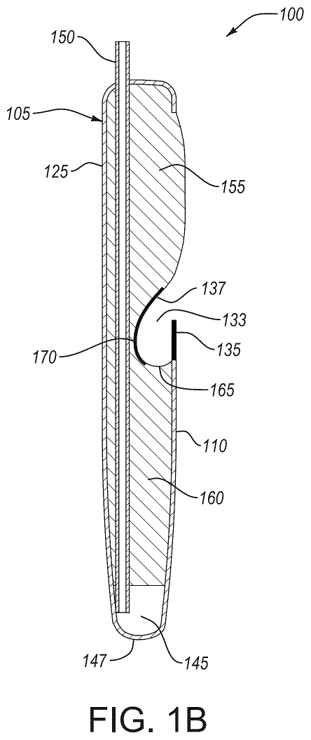

FIG. 1A is a front plan view of a urine collection device 100, according to an embodiment. Conventional urine collection devices may be configured to collect urine from only one of (1) a protruding penis or (2) a male having a buried penis and/or a female. The urine collection device 100 is an example of a urine collection device 100 that is configured to receive fluids from a male with either a buried or a protruding penis, or a female. Thus, urine collection device 100 improves upon conventional urine collection device because the urine collection device 100 may be suitable for effective use with multiple genders and/or anatomical features.

The urine collection device 100 includes a fluid impermeable barrier 105 having a generally tubular elongated portion 110 and a second portion 125. The elongated portion 110 includes a width W₁ and a length L₁. The second portion 125 includes a width W₂ that is greater than the width W₁ of the elongated portion 110 and a length L₂. The second portion 125 may narrow from the width W₂ to the elongated portion 110. For example, the second portion 125 may be generally triangular and narrow towards the elongated portion 110. In some embodiments, the width W₂ of the second portion 125 is at least 1.5 or 2.0 times greater than the width W₁ of the elongated portion 110. For example, the width W₁ of the elongated portion 110 may be about 0.5 inches to about 1 inch and the width W₂ of the second portion 125 may be at least about 1.5 inches or at least about 2 inches. The length L₁ of the elongated portion 110 may be less than, greater than, or substantially equal to the length L₂ of the second portion 125. For example, length L₁ of the elongated portion 110 may be about 0.5 to about 1.5 times the length L₂ of the second portion 125.

The second portion 125 of the fluid impermeable barrier 105 may include a substantially flat or planar area. That is, the second portion 125 of the fluid impermeable barrier 105 may include a substantially flat back side and front side defining an opening 120 having a substantially planar profile. The fluid impermeable barrier 105 also may include or define an aperture 115 on the second portion 125 distal to the elongated portion 110. The aperture 115 is configured to receive a conduit 150 therethrough. The fluid impermeable barrier 105 also at least partially defines a chamber (e.g., interior region). The opening 120 is formed in and extends through the fluid impermeable barrier 105, thereby enabling fluids to enter the chamber from outside of the urine collection device 100. The opening 120 can be configured to be positioned adjacent to a female urethra, positioned adjacent to the skin of a female over the urethra, or positioned adjacent to the skin over the penis of a male having a buried penis. The opening 120 can include a maximum lateral dimension D₁ that is at least 1.5 times greater than the width W₁ of the elongated portion 110. For example, the opening 120 can include a maximum lateral dimension D₁ that is at least about 1.5 inches, at least about 2 inches, at least about 2.5 inches, at least about 3 inches, about 1.5 inches to about 2.5 inches, or about 2.5 inches to about 3.5 inches. The opening 120 can include a maximum lateral dimension D₁ that is at least 1.5 times greater than the width W₁ of the elongated portion 110, which allows for easier positioning and securement of the urine collection device 100 over a buried penis, with the elongated portion 110 extending between the thighs and pelvis for more secure positioning of the urine collection device 100 against the individual. With the urine collection device 100 positioned proximate to the female urethra, positioned adjacent to the skin of a female over the urethra, or positioned adjacent to the skin over the penis of a male having a buried penis, urine may enter the interior region or chamber of the urine collection device 100 via the opening 120. Accordingly, the urine collection device 100 is configured to receive the fluids into the chamber via the opening 120.

The fluid impermeable barrier 105 may also temporarily store the fluids in the chamber. As such, the fluid impermeable barrier 105 substantially prevents the fluids from exiting the portions of the chamber that are spaced from the opening 120. The fluid impermeable barrier 105 is flexible, allowing the urine collection device 100 to bend or curve when positioned against the body of a wearer. For example, the fluid impermeable barrier 105 can be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer, oil, another suitable material, or combinations thereof.

The urine collection device 100 also includes a penis pocket 133 sized and dimensioned to receive at least a head of a penis within the pocket 133. The pocket 133 may extend from the opening 120 toward the elongated portion 110 of the fluid impermeable barrier 105. The pocket 133 may be defined at least partially by an outer wall 135 positioned on the outside of the urine collection device 100 and an inner wall positioned between the outer wall and at least a portion of the fluid permeable body 130. In some embodiments the pocket 133 is defined at least partially by a mesh outer wall 135 and an inner wall 137. The outer wall 135, such as a mesh outer wall, may be flexible or elastic, and may extend across a lower portion of the opening 120 proximate to the elongated portion 110 of the fluid impermeable barrier 105. For example, the opening 120 may narrow towards the elongated portion 110, and the outer wall 135 may cover at least a portion of the opening 120 that narrows towards the elongated portion 110. Accordingly, the opening 120 may include a portion extending from the second portion 125 of the fluid impermeable barrier 105 towards the elongated portion 110 that has a width that is less than the maximum lateral dimension D₁ of the opening 120. In other embodiments, the outer wall may include a fluid impermeable outer wall.

Turning to FIG. 1B, which shows a cross-sectional view of the urine collection device taken along line A-A of FIG. 1A, the pocket 133 also is partially defined by the inner wall 137 extending into the chamber of the fluid impermeable barrier 105. Accordingly, the pocket 133 may extend at least partially into the chamber defined by the fluid impermeable barrier 105. The inner wall 137 may include a fluid impermeable inner wall or a fluid permeable inner wall. The inner wall 137 may extend from a lower portion of the opening 120 towards the elongated portion 110. In some embodiments, the inner wall 137 extends from a lower portion of the opening 120 to a hole 165 or base of the pocket 133 positioned beyond the opening 120. For example, the inner wall 137 may extend from a lower portion of the opening 120 to the hole 165 positioned within the chamber defined by the elongated portion 110 of the fluid impermeable barrier 105. In other embodiments, the inner wall 137 is substantially planar with the fluid permeable body 130 exposed through the opening 120, such that there is no recess in the fluid permeable body 130. In some embodiments, the fluid permeable body is the inner wall 137 defining at least a portion of the pocket 133 and the fluid impermeable inner wall is absent. For example, in some embodiments, the pocket 133 is at least partially defined by a mesh outer wall or a fluid impermeable outer wall and a fluid permeable inner wall of the fluid permeable body 130.

When used, an individual may position at least the head of the penis within the pocket 133 such that a portion of the head of the penis interfaces the inner wall 137, a portion of the head of the penis interfaces the outer wall 135, and the urethra of the penis is positioned proximate to the hole 165 or base of the pocket 133. With the urethra of the penis positioned proximate to the hole 165, fluids discharged from the penis exit the pocket 133 through the hole 165 and into the chamber defined by the fluid impermeable barrier 105.

The urine collection device 100 includes a fluid permeable body 130 or layer disposed in the chamber. The fluid permeable body 130 can cover or extend across at least a portion (e.g., all) of the opening 120. The portion of the fluid permeable body 130 covering or extending across at least a portion of the opening 120 may be substantially flat or planar. Accordingly, the second portion 125 fluid impermeable barrier 105 may define an opening 120 on the front side having a substantially planar profile with the fluid permeable body 130 positioned therein.

The fluid permeable body 130 can be configured to wick any fluid away from the opening 120, thereby preventing the fluid from escaping the chamber of the fluid impermeable barrier 105. The fluid permeable body 130 also can wick the fluid generally towards an interior of the chamber, as discussed in more detail below. A portion of the fluid permeable body 130 can define a portion of an outer surface of the urine collection device 100. Specifically, the portion of the fluid permeable body 130 defining the portion of the outer surface of the urine collection device 100 can be the portion of the fluid permeable body 130 exposed by the opening 130 defined by the fluid impermeable barrier 105 that contacts the user.

The fluid permeable body 130 can include any material that can wick or allow the fluid to flow therethrough. The permeable properties referred to herein can be wicking, capillary action, diffusion, or other similar properties or processes, and are referred to herein as “permeable” and/or “wicking.” Such “wicking” may exclude absorption into the wicking material.

The fluid permeable body 130 can include a one-way fluid movement fabric. As such, the fluid permeable body 130 can remove fluid from the area around the buried penis or female urethra, thereby leaving the area and urethra dry. The fluid permeable body 130 can enable the fluid to flow generally towards a reservoir 145 of void space formed within the chamber. The reservoir 145 may be positioned within a distal end 147 of the elongated portion 110 distal to the second portion 125. The fluid permeable body 130 can include a porous or fibrous material, such as hydrophilic polyolefin. In some embodiments, the fluid permeable body 130 consists of or consists essentially of a porous or fibrous material, such as hydrophilic polyolefin. Examples of polyolefin that can be used in the fluid permeable body 130 include, but are not limited to, polyethylene, polypropylene, polyisobutylene, ethylene propylene rubber, ethylene propylene diene monomer, or combinations thereof. The porous or fibrous material may be extruded into a substantially shape to fit within the chamber of the fluid impermeable barrier 105. The fluid permeable body 130 may be shaped generally complementary to the shape of the fluid impermeable barrier 105. For example, the fluid permeable body 130 may include at least a substantially flat or planar portion that extends across at least a portion of the opening 120. The fluid permeable body 130 also may additionally or alternatively include a substantially flat or planar portion that interfaces the back side of the fluid impermeable barrier 105 opposite of the opening 120. Moreover, the fluid permeable body 130 can be manufactured according to various manufacturing methods, such as molding, extrusion, or sintering. The fluid permeable body 130 can include varying densities or dimensions.

In some embodiments, the fluid permeable body 130 can include two or more layers of fluid permeable materials and include no more than two layers of material between the opening 130 and the conduit 150 positioned within the fluid permeable body 130. For example, the urine collection device 100 can include a fluid permeable membrane covering or wrapped around a fluid permeable support of the fluid permeable body 130, with both the fluid permeable membrane and the fluid permeable support being disposed in the chamber of the fluid impermeable barrier 105. The fluid permeable membrane can cover or extend across at least a portion (e.g., all) of the opening 120. The fluid permeable membrane and the fluid permeable support can be configured to wick any fluid away from the opening 120, thereby preventing the fluid from escaping the chamber. The permeable properties referred to herein can be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 120. The material of the fluid permeable membrane and the fluid permeable support also can include natural fibers. In such examples, the material may have a coating to prevent or limit absorption of fluid into the material, such as a water repellent coating. Such “wicking” may not include absorption into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material.

The fluid permeable membrane can also wick the fluid generally towards an interior of the chamber, as discussed in more detail below. The fluid permeable membrane can include any material that can wick the fluid. For example, the fluid permeable membrane can include fabric, such as a gauze (e.g., a silk, linen, polymer based materials such as polyester, or cotton gauze), nylon, another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the urine collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 130 includes a fluid permeable support including a porous nylon structure (e.g., spun nylon fibers) and a fluid permeable membrane including gauze about or over the porous nylon structure.

The fluid permeable body 130 may be shaped generally complementary to the shape of the fluid impermeable barrier 105. In some embodiments, the fluid permeable body 130 includes an elongated portion 160 positioned within the elongated portion 110 of the fluid impermeable barrier 105 and a second portion 155 positioned within the second portion 125 of the fluid impermeable barrier 105. The fluid permeable body 130 may include a recess 170 around at least a portion of the pocket 133. For example, a portion of the elongated portion 160 and a portion of the second portion 155 of the fluid permeable body 130 may include a recess complementary or proximate to the inner wall 137. In some embodiments, the portion of the fluid permeable body 130 defining the recess also defines the inner wall 137. The recess 170 is sized and dimensioned to receive at least a portion of the head of the penis therein to allow insertion of the head of the penis into the pocket 133. The recess 170 in the fluid permeable body 130 also positions a portion of the fluid permeable body proximate to the hole 165 in the pocket 133.

The elongated portion 160 and the second portion 155 of the fluid permeable body 130 may be continuously connected to one another, or a gap or layer may be positioned between the elongated portion 160 and the second portion 155 of the fluid permeable body 130. In some embodiments, a fluid impermeable barrier (not shown) separates the second portion 155 from the elongated portion 160 of the fluid permeable body. This fluid impermeable barrier is positioned to prevent urine or other fluids collected through the opening 120 from exiting through the pocket 133. Instead, these fluids may be collected directly from the second portion 155 of the fluid permeable body 130 through, for example, perforations in the conduit 150 positioned in the second portion 155.

The conduit 150 (e.g., a tube) extends through the aperture 115 (shown in FIG. 1A) into the chamber defined by the fluid impermeable barrier 105. For example, the conduit 150 may extend through the aperture 115 to provide fluid communication between the conduit 150 and the reservoir 145. The conduit 150 may extend into the reservoir 145, as shown in FIG. 1B, or the conduit 150 may be generally flush with or recessed from the portion of the fluid permeable support 150 defining the reservoir 145. The conduit 150 may extend through or be positioned proximate to the second portion 155 and the elongated portion 160 of the fluid permeable body 130 in the chamber defined by the fluid impermeable barrier 105. The conduit 150 provides fluid communication between the chamber and a fluid storage container (not shown) or a portable vacuum source (not shown). For example, the conduit 150 may directly or indirectly fluidly couple the chamber and/or the reservoir 145 with the fluid storage container or the portable vacuum source. With the conduit 150 positioned within the fluid permeable body 130, when the urine collection device 100 is secured to a user with any of a number of securing devices, fluids received in the chamber can be removed through the conduit 150.

The fluid impermeable barrier 105 can store fluids in the reservoir 145 therein. The reservoir 145 is an unoccupied portion of the chamber and is void of other material (e.g., the fluid permeable body 130 and the fluid impermeable barrier 105 are absent from the reservoir 145). In some embodiments, the reservoir 145 is defined at least partially by the fluid permeable body 130 and the fluid impermeable barrier 105. The reservoir 145 may be disposed in any portion of the interior region of the chamber. For example, the fluid reservoir 145 may be positioned in the distal end 147 of the elongated portion 110. In the illustrated embodiment, the reservoir 145 is defined by at least a portion of an end of the elongated portion 160 the fluid permeable body 130 and the distal end 147 of the elongated portion 110 of the fluid impermeable barrier 105.

In use, the urine collection device 100 is not limited to a single gender or anatomical features, but advantageously may be used with a variety of anatomies and/or genders. For example, the opening 120 of the urine collection device 100 may be positioned over a buried penis of a male or proximate to a female urethra to collect discharged urine or other bodily fluids through the opening and into the fluid permeable body 130. Alternatively, at least a portion of the head of the penis may be inserted into the pocket 133 of the urine collection device 100 to collect discharged urine or other bodily fluids through the opening 165 and into the fluid permeable body 130. The discharged urine or other bodily fluids may be wicked through the fluid permeable body 130 to the conduit 150 for removal from the urine collection device 100 through the conduit 150. The discharged urine or other bodily fluids may be wicked through the fluid permeable body 130 to the reservoir 145 for removal from the urine collection device 100 through the conduit 150. The discharged urine or other bodily fluids may be wicked through the fluid permeable body 130 to perforations in the conduit 150 for removal from the urine collection device 100 through the conduit 150.

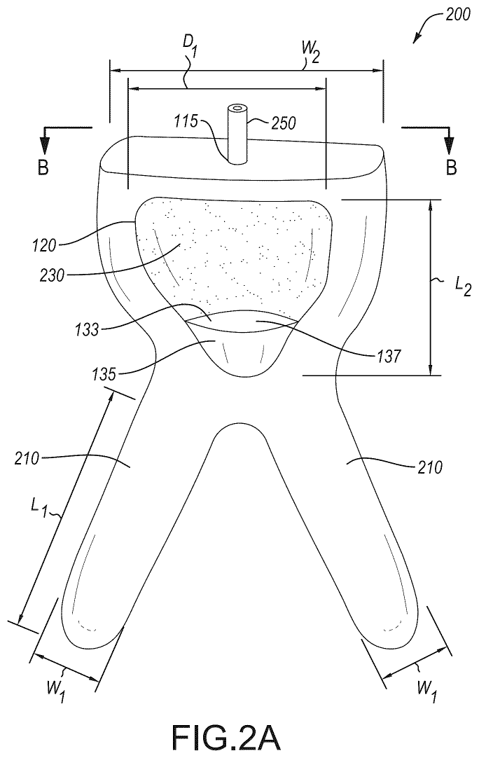

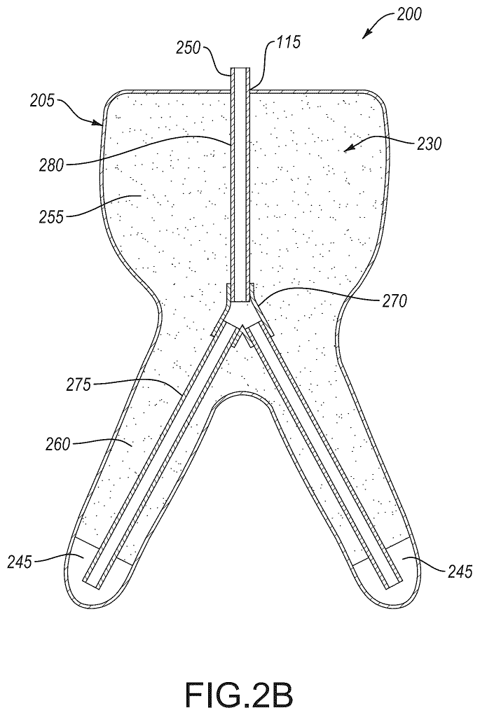

Turning to FIGS. 2A-2B, a urine collection device 200 may include a fluid impermeable barrier 205 having two elongated portions 210 and an upper or second portion 125. Unless otherwise noted, the urine collection device 200 may include any of the elements or features described above in relation to the urine collection device 100, such as the aperture 115, the opening 120, the pocket 133, the outer wall 135, the inner wall 137, the width W₂, the maximum lateral dimension D₁, and/or the length L₂. Moreover, the fluid impermeable barrier 205 may include any of the materials of the fluid impermeable barrier 105 described in relation to the urine collection device 100. In some embodiments, each of the two elongated portions 210 may include a width W₁ similar or equal to the width W₁ of the urine collection device 100.

The two elongated portions 210 of the urine collection device 200 may be angled relative to one another. For example, the two elongated portions 210 may be angled to form a V-shaped configuration that allows the scrotum and testicles of a user to be positioned or held between two elongated portions 210. This configuration allows the user to lay on his side while wearing or using the urine collection device 200. The angle between the two elongated may be about 30 degrees to about 150 degrees, about 30 degrees to about 60 degrees, about 60 degrees to about 90 degrees, about 90 degrees to about 120 degrees, or about 120 degrees to about 150 degrees.

The urine collection device 200 also includes a fluid permeable body 230 or layer disposed in the chamber defined by the fluid impermeable barrier 205. The fluid permeable body 230 may include any of the materials of the fluid permeable body 230 described in relation to the urine collection device 100. The fluid permeable body 230 can cover or extend across at least a portion (e.g., all) of the opening 120. The portion of the fluid permeable body 230 covering or extending across at least a portion of the opening 120 may be substantially flat or planar. Accordingly, the second portion 225 fluid impermeable barrier 205 may define an opening 120 on the front side having a substantially planar profile with the fluid permeable body 230 positioned therein.

The fluid permeable body 230 may be shaped generally complementary to the shape of the fluid impermeable barrier 205. In some embodiments, the fluid permeable body 230 includes two elongated portions 260 positioned within the elongated portions 210 of the fluid impermeable barrier 205 and a second portion 255 positioned within the second portion 225 of the fluid impermeable barrier 205. The fluid permeable body 230 may include a recess around at least a portion of the pocket 133. For example, at least one of a portion of the elongated portions 260 or a portion of the second portion 255 of the fluid permeable body 230 may include a recess complementary or proximate to the inner wall 137. The recess is sized and dimensioned to receive at least a portion of the head of the penis therein to allow insertion of the head of the penis into the pocket 133. The recess in the fluid permeable body 230 also positions a portion of the fluid permeable body proximate to a hole in the pocket 133.

The elongated portions 260 and the second portion 255 of the fluid permeable body 230 may be continuously connected to one another, or a gap or layer may be positioned between the elongated portions 260 and the second portion 255 of the fluid permeable body 230. In some embodiments, a fluid impermeable barrier (not shown) separates the second portion 255 from the elongated portions 260 of the fluid permeable body. This fluid impermeable barrier is positioned to prevent urine or other fluids collected through the opening 120 from exiting through the pocket 133. Instead, these fluids may be collected directly from the second portion 255 of the fluid permeable body 230 through, for example, perforations in the conduit 150 positioned in the second portion 255.

The conduit 250 (e.g., a tube) extends through the aperture 115 into the chamber defined by the fluid impermeable barrier 205. For example, the conduit 250 may extend through the aperture 115 to or partially into the reservoirs 245 in the two elongated portions 210. The conduit 250 may include two first portions 275 each positioned in a different elongated portion 210 of the fluid impermeable barrier 205. The conduit 250 also may include a second portion 280 positioned in the second portion 125 of the urine collection device 200. A Y connector 270 may connect the second portion 280 of the conduit 250 with the two first portions 275 of the conduit 250, thereby providing fluid communication between the two reservoirs 245 in the two elongated portions 210 with the second portion 280 of the conduit 250. The second portion 280 of the conduit 250 may extend through or be positioned proximate to the second portion 255 and the elongated portions 260 of the fluid permeable body 230 in the chamber defined by the fluid impermeable barrier 205. The conduit 250 provides fluid communication between the chamber and a fluid storage container (not shown) or a portable vacuum source (not shown). For example, the conduit 250 may directly or indirectly fluidly couple the chamber and/or the reservoirs 245 with the fluid storage container or the portable vacuum source. With the conduit 250 positioned within the fluid permeable body 230, when the urine collection device 100 is secured to a user with any of a number of securing devices, fluids received in the chamber can be removed through the conduit 250.

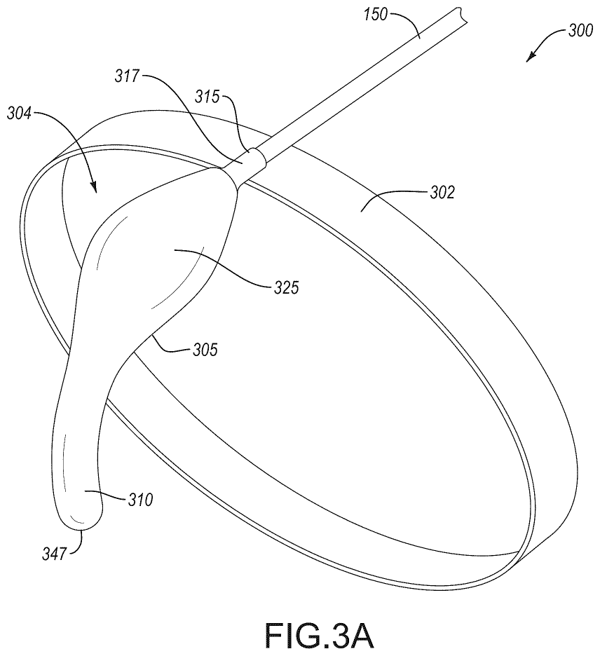

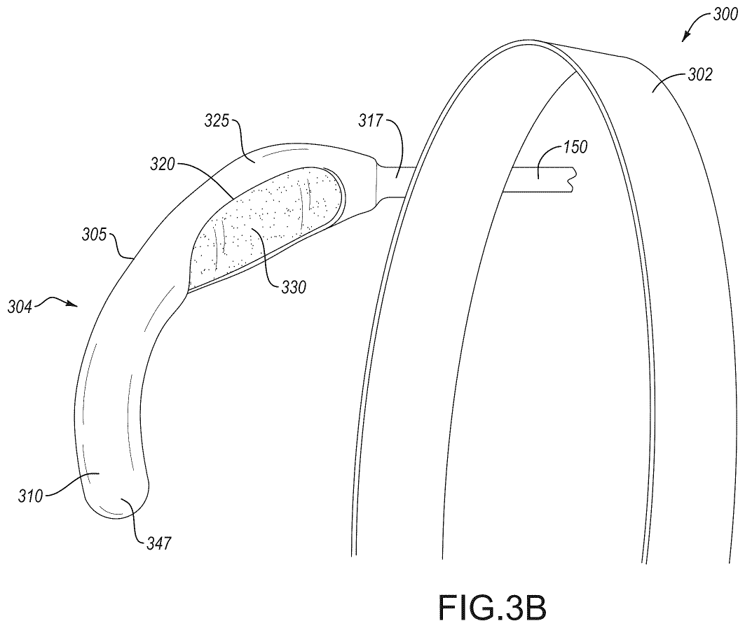

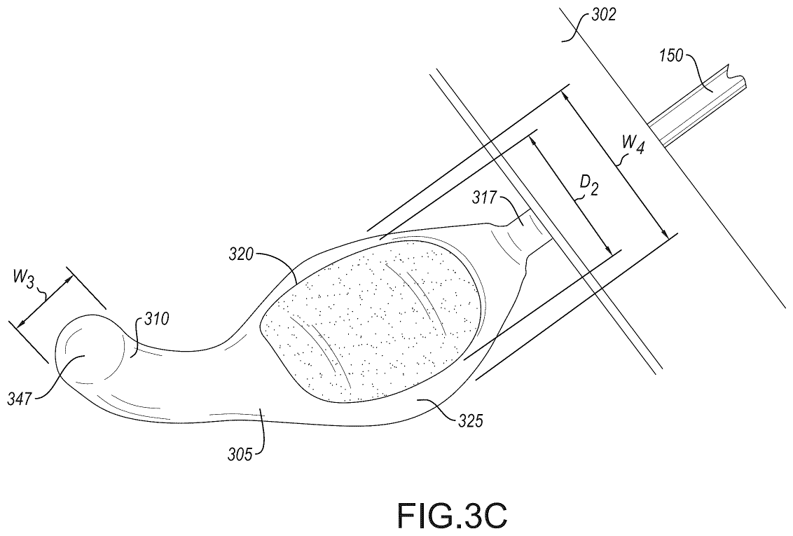

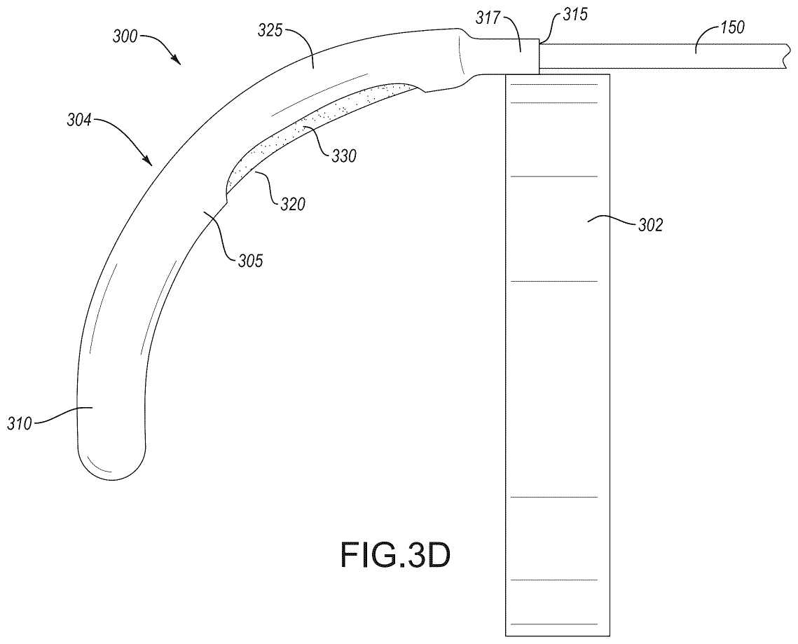

Many male patients that may benefit from a urine collection system have a buried penis or a penis with insufficient length to support a conventional male urine collection device. Accordingly, a urine collection device having an elongated portion for positioning between the legs and also a larger opening for collecting urine would improve urine collection for these individuals. FIG. 3A is an isometric view of a urine collection system 300 including a urine collection device 304 secured to a belt 302. FIG. 3B is a partial rear view of the urine collection system 300 with the urine collection device 304 secured to the belt 302. FIG. 3C is a close-up view of the urine collection device 304 secured to the belt 302. FIG. 3D is a side view of the urine collection system 300 with the urine collection device 304 secured to the belt 302. References to FIGS. 3A-3D are made below.

The urine collection device 304 is an example of a urine collection device 304 that is configured to receive fluids from a male with a buried penis or a penis of limited length, or a female. The urine collection device 304 of the urine collection system 300 is secured to a belt 302. The belt 302 may include an elastic belt sized to fit around the waist or a leg of a wearer to position the opening 320 of the urine collection device 304 over the urethra of the individual wearing the urine collection system 300. The urine collection device 304 may be fixedly or detachably secured to the belt 302. In some embodiments, the urine collection device 304 is secured to the belt 302 with one or more of an adhesive, stitching, magnets, or a hook and loop fastener, such as VELCRO®. Although not shown in FIGS. 1A-2B, the belt 302 may be used with the urine collection device 100 or 200. For example, the urine collection device 100 or 200 may be secured to the belt 302 as described in relation to the urine collection device 304.

The urine collection device 304 includes a fluid impermeable barrier 305 having a generally tubular elongated portion 310 and a second portion 325. The elongated portion 310 includes a width W₃. The width W₃ of the elongated portion may be generally continuous until the elongated portion 310 tapers at a rounded distal end. The second portion 325 includes a maximum width W₄ that is greater than the width W₃ of the elongated portion 310, and the second portion 325 may narrow from the maximum width W₄ to the elongated portion 310. For example, the second portion 325 may include a generally oval profile, with the oval being elongated between the neck 317 and the elongated portion 310. In some embodiments, the maximum width W₄ of the second portion 325 is at least 1.5 or 2.0 times greater than the width W₃ of the elongated portion 310. For example, the width W₃ of the elongated portion 310 may be about 0.5 inches to about 1 inch and the maximum width W₄ of the second portion 325 may be at least about 1.5 inches or at least about 2 inches.

The fluid impermeable barrier 305 also may include a neck 317 defining an aperture 315 on the second portion 325 distal to the elongated portion 310. The neck 317 may be secured to the belt 302, and the aperture 315 is configured to receive a conduit 150 therethrough. The fluid impermeable barrier 305 also at least partially defines a chamber (e.g., interior region). The opening 320 is formed in and extends through the fluid impermeable barrier 305, thereby enabling fluids to enter the chamber from outside of the urine collection device 304. The opening may be generally oval in shape complementary to the second portion 325. The opening 320 can be configured to be positioned adjacent to a female urethra, positioned adjacent to the skin of a female over the urethra, or positioned adjacent to the skin over the penis of a male having a buried penis. The opening 320 can include a lateral dimension D₂ that is at least about 1.5 times greater than the width W₃ of the elongated portion 310. For example, the opening 320 can include a lateral dimension D₂ that is at least about 1.5 inches. A urine collection device having opening 320 can include a lateral dimension D₂ that is at least about 1.5 times greater than the width W₃ of the elongated portion 310 allows for easier positioning and securement of the urine collection device 304 over a buried penis, with the elongated portion 310 extending between the thighs and pelvis for more secure positioning of the urine collection device 304 against the individual. With the urine collection device 304 positioned proximate to the female urethra, positioned adjacent to the skin of a female over the urethra, or positioned adjacent to the skin over the penis of a male having a buried penis, urine may enter the interior region or chamber of the urine collection device 304 via the opening 320. Accordingly, the urine collection device 304 is configured to receive the fluids into the chamber via the opening 320.

The fluid impermeable barrier 305 may also temporarily store the fluids in the chamber. As such, the fluid impermeable barrier 305 substantially prevents the fluids from exiting the portions of the chamber that are spaced from the opening 320. The elongated portion 310 and the second portion 325 of fluid permeable barrier 305 may be shaped to generally form an arc (shown in FIG. 3D). The fluid permeable barrier 305 may include a length of about 6 to 8 inches from the neck 317 to the distal end 347. The fluid impermeable barrier 305 is flexible, allowing the urine collection device 304 to bend or curve when positioned against the body of a wearer. For example, the fluid impermeable barrier 305 can be formed of any suitable fluid impermeable materials described above in relation to the fluid impermeable barrier 105.

The urine collection device 304 includes a fluid permeable body 330 or layer disposed in the chamber. The fluid permeable body 330 can cover or extend across at least a portion (e.g., all) of the opening 320. The portion of the fluid permeable body 330 covering or extending across at least a portion of the opening 320 may be substantially flat or arced with the arc of the fluid impermeable barrier 305. Accordingly, the second portion 325 fluid impermeable barrier 305 may define an opening 320 on the front side having a substantially planar profile or an arced profile with the fluid permeable body 330 positioned therein.

The fluid permeable body 330 can be configured to wick any fluid away from the opening 320, thereby preventing the fluid from escaping the chamber of the fluid impermeable barrier 305. The fluid permeable body 330 also can wick the fluid generally towards an interior of the chamber. A portion of the fluid permeable body 330 can define a portion of an outer surface of the urine collection device 304. Specifically, the portion of the fluid permeable body 330 defining the portion of the outer surface of the urine collection device 304 can be the portion of the fluid permeable body 330 exposed by the opening 330 defined by the fluid impermeable barrier 305 that contacts the user. The fluid permeable body 330 can include any material described above in relation to the fluid permeable body 130.

The fluid permeable body 330 may be shaped generally complementary to the shape of the fluid impermeable barrier 305. In some embodiments, the fluid permeable body 330 includes an elongated portion positioned within the elongated portion 310 of the fluid impermeable barrier 305 and a second portion positioned within the second portion 325 of the fluid impermeable barrier 305. The second portion may be generally oval shaped complementary to the oval shape of the second portion 325. The elongated portion and the second portion of the fluid permeable body 330 may be continuously connected to one another.

The conduit 150 extends through the aperture 315 into the chamber defined by the fluid impermeable barrier 305. For example, the conduit 150 may extend through the aperture 315 to or partially into the reservoir in the distal end 347 of the elongated portion 310. In use, the opening 320 of the urine collection device 304 may be positioned over a buried penis of a male or proximate to a female urethra to collect discharged urine or other bodily fluids through the opening 320 and into the fluid permeable body 330. The discharged urine or other bodily fluids may be wicked through the fluid permeable body 330 to the conduit 150 for removal from the urine collection device 304 through the conduit 150. The discharged urine or other bodily fluids may be wicked through the fluid permeable body 330 to the reservoir for removal from the urine collection device 304 through the conduit 150.

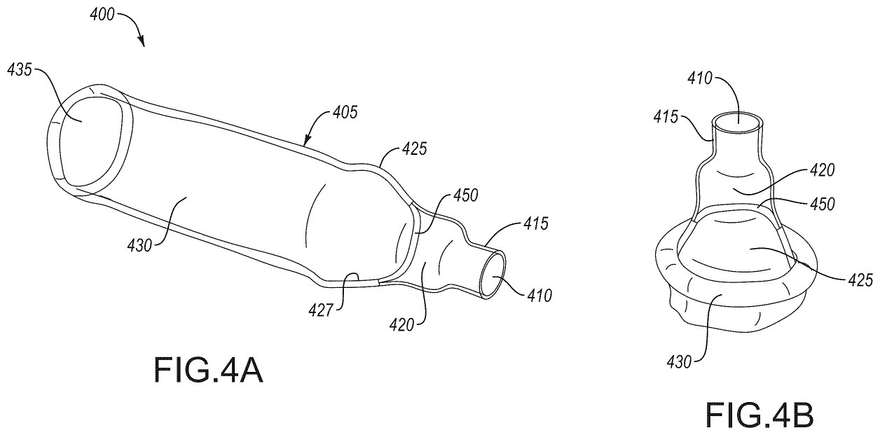

A urine collection device according to this disclosure also may include an improved male external catheter. FIG. 4A shows an isometric view of a urine collection device 400 according to an embodiment. The urine collection device 400 includes a fluid impermeable barrier 405 defining a chamber and having an elongated portion 430, a receptacle 425, a port include a neck 415 defining an aperture 410 distal to the elongated portion 430, and an opening 435 distal to the receptacle 425. The fluid impermeable barrier 405 may include any materials described above in relation to the urine collection devices 100, 200, or 300. In some embodiments, the fluid impermeable barrier 405 includes a rubber or silicone material.

The elongated portion 430 may form a sheath sized to secure the urine collection device 400 to the shaft of a penis with at least a portion of the shaft of the penis being inside the elongated portion. In some embodiments, the elongated portion 430 may unroll from a retracted position (shown in FIG. 4B) to an extended position (shown in FIG. 4A) around the shaft of the penis.

The receptacle 425 is proximate to the elongated portion 430 and may be sized and dimensioned generally frustoconical to receive at least a portion of a head of a penis therein. The receptacle 425 may include an inner surface that contacts at least a portion of a head of a penis when a penis is positioned within the urine collection device.

The receptacle 425 also may include a fluid permeable body 450 positioned within the chamber defined by the fluid impermeable barrier 405 to be proximate to the urethra on the head of the penis positioned at least partially within the receptacle 425. For example, the receptacle 425 may include a wide end and a narrow end, and the fluid permeable body 450 may be positioned at the narrow end of the receptacle. In some embodiments, the fluid permeable body defines the inner wall of the receptacle 425 such that substantially all of the head of the penis interfaces the fluid permeable body 450 and not the fluid impermeable barrier 405. The fluid permeable body 450 may be secured to the fluid impermeable barrier 405 with an adhesive. The fluid permeable body 450 is configured to wick urine or other fluids discharged from the urethra away from the head of the penis. The fluid permeable body 450 may include any of the materials described above in relation to the fluid permeable bodies 130, 230, or 330.

The fluid permeable body 450 is positioned within the fluid impermeable barrier 405 to wick discharged urine from the receptacle 425 to a reservoir 420. The reservoir 420 may be an unoccupied portion of the urine collection device 400 that is void of other material and between the neck 415 and receptacle 425. The aperture 410 defined by the neck 415 is configured to receive a conduit (not shown) therethrough. The conduit provides fluid communication between the reservoir 420 and a fluid storage container (not shown) or a portable vacuum source (not shown). For example, the conduit may directly or indirectly fluidly couple the reservoir with the fluid storage container or the portable vacuum source. With the conduit positioned within the fluid permeable body neck 415, when the urine collection device 400 is secured to the penis, fluids received in the reservoir 420 can be removed through the conduit.

An individual may receive the urine collection device 400 with the elongated portion 430 in a retracted position as shown in FIG. 4B before securing the urine collection device to the penis. The urine collection device 400 may be secured to the penis by inserting the penis through the opening 435 and positioning at least a portion of the head of the penis in the receptacle 425. The elongated portion 430 may then be unrolled over the shaft of the penis until the elongated portion 430 is in the extended position shown in FIG. 4A.

With the head of the penis at least partially positioned in the receptacle 425 and the elongated portion 430 securing the urine collection device 400 to the penis, urine discharged from the penis may be withdrawn from the urine collection device 400. When urine is discharged from the urethra of the penis, the fluid permeable body 430 wicks the urine away from the urethra. The urine may be wicked to the reservoir 420, before being pulled from the reservoir 420 by a conduit secured to the neck 415. The configuration of the urine collection device 400 may prevent urinary tract infections associated with internal catheters, while also preventing dermatitis associated with adult diapers.

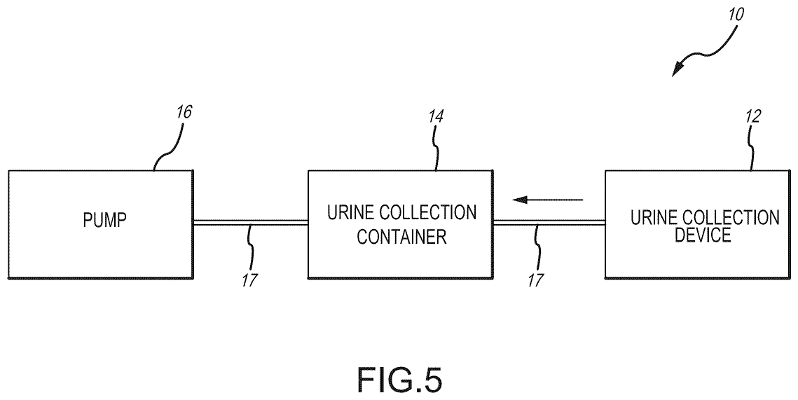

FIG. 5 is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection device disclosed herein), a urine collection container 14, and a pump 16 (or vacuum source). The fluid collection device 10, the urine collection container 14, and the pump 16 may be fluidly coupled to each other via one or more conduits 17. For example, fluid collection device may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 10 may be removed from the fluid collection device 10 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle or cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 or vacuum source may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

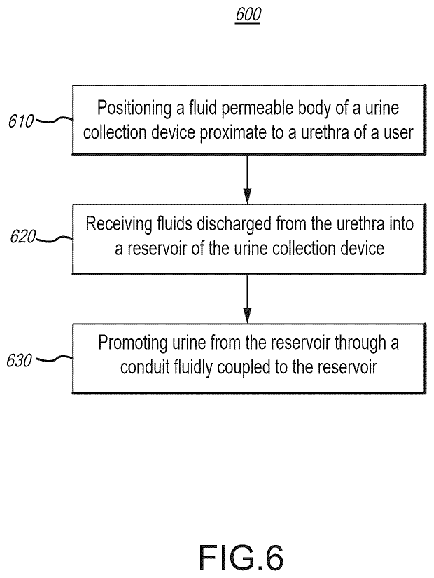

FIG. 6 is a flow diagram of a method 600 for collecting urine, according to an embodiments. The method 600 includes an act 610 of positioning a fluid permeable body of a urine collection device at least proximate to a urethra of a user. The fluid permeable body may extend at least partially across an opening defined by a fluid impermeable barrier of the urine collection device. The urine collection device may include any of the urine collection device described herein. The method 600 also includes an act 620 of receiving fluids discharged from the urethra into a reservoir void of material of the urine collection device. The reservoir may be at least partially defined by a portion of the fluid permeable body and a portion of the fluid impermeable barrier. The method 600 also includes an act 630 of promoting urine from the reservoir through a conduit fluidly coupled to the reservoir.

In some embodiments, the act 610 of positioning a fluid permeable body of a urine collection device at least proximate to a urethra of a user includes positioning a substantially planar or concave surface of the fluid permeable body extending across an opening of the fluid impermeable barrier at least proximate to the urethra of the user. The method 600 also may include an act of positioning a waist belt around a waist of the user, the waist belt being secured to the fluid impermeable barrier of the urine collection device and the urine collection device having an arched elongated portion.

In some embodiments, the act 610 of positioning a fluid permeable body of a urine collection device at least proximate to a urethra of a user includes inserting at least a head of a penis of the user into a pocket positioned on or adjacent to the fluid permeable body to extend from a portion of the opening of the fluid impermeable barrier towards the reservoir. Inserting at least a head of a penis of the user into a pocket positioned on or adjacent to the fluid permeable body may include inserting at least the head of the penis of the user into a pocket extending into a recess formed in the fluid permeable body. Inserting at least a head of a penis of the user into a pocket positioned on or adjacent to the fluid permeable body may include inserting at least the head of the penis of the user into the pocket at least partially defined by a material extending across a portion of the opening that holds at least the head of the penis between the material and the fluid permeable body. The method 600 also may include an act of positioning a scrotum of the user between two elongated portions of the fluid impermeable barrier.

In some embodiments, the act 610 of positioning a fluid permeable body of a urine collection device at least proximate to a urethra of a user may include inserting at least a head of a penis into a receptacle of the urine collection device with at least a portion of an inner surface of the receptacle contacting the head of the penis and at least a portion of the fluid permeable body at least partially defining the receptacle. Inserting at least a head of a penis into a receptacle of the urine collection device may include inserting the head of the penis into frustoconical region of the fluid impermeable barrier at least partially defining the receptacle and inserting a shaft of the penis into a sheath of the fluid impermeable barrier connected to the frustoconical region of the fluid impermeable barrier, the sheath securing the urine collection device to the penis. In some embodiments, the method 600 also includes an act of connecting the conduit to a port on the urine collection device, the reservoir being positioned between the port and the fluid permeable body.

The acts of the method 600 described above are for illustrative purposes. For example, the acts of the method 600 can be performed in different orders, split into multiple acts, modified, supplemented, or combined. In an embodiment, one or more of the acts of the method 600 can be omitted from the method 600. Any of the acts of the method 600 can include using any of the urine collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A urine collection device, comprising:
a fluid impermeable layer including a first end region having a first width and a second end region having a second width that is at least about 2 times greater than the first width of the first end region such that the fluid impermeable layer narrows between the second end region and the first end region;
one or more fluid permeable layers secured to the fluid impermeable layer, at least one fluid permeable layer of the one or more fluid permeable layers positioned to be at least proximate to a urethra of a subject when the urine collection device is secured to the subject; and
a conduit positioned between at least a portion of the one or more fluid permeable layers and the fluid impermeable layer, the conduit extending from an inlet at least proximate to the first end region of the fluid impermeable layer to the second end region of the fluid impermeable layer, wherein the conduit is configured to couple to a vacuum source effective to pull urine discharged into the urine collection device into the inlet and through the conduit for removal from the urine collection device.

2. The urine collection device of claim 1, wherein at least a portion of the at least one fluid permeable layer of the one or more fluid permeable layers positioned to be at least proximate to the urethra of the subject forms a substantially concave surface on the urine collection device.

3. The urine collection device of claim 2, wherein the at least a portion of the at least one fluid permeable layer of the one or more fluid permeable layers includes a wicking material forming the substantially concave surface positioned to be at least proximate to the urethra of the subject.

4. The urine collection device of claim 1, further comprising a reservoir of void space at the first end region of the fluid impermeable layer and configured to hold at least some of the urine discharged into the urine collection device, the inlet of the conduit being positioned at least proximate to the reservoir.

5. The urine collection device of claim 4, wherein the one or more fluid permeable layers include at least a wicking material configured to direct the urine towards the reservoir and the inlet of the conduit.

6. The urine collection device of claim 1, wherein the fluid impermeable layer defines an aperture at the second end region and the conduit extends through the aperture.

7. The urine collection device of claim 1, wherein the fluid impermeable layer includes a fluid impermeable film and at least one permeable layer of the one or more permeable layers includes a wicking material.

8. The urine collection device of claim 7, wherein the wicking material forms at least a portion of a front side of the urine collection device and the fluid impermeable film forms a rear side of the urine collection device.

9. A urine collection device, comprising:
a first region of fluid impermeable material at a first end region of the urine collection device, the first end region having a first width and the first region of fluid impermeable material defining a reservoir of void space in the first end region of the urine collection device;
a second region of fluid impermeable material at a second end region of the urine collection device and narrowing in width as the second region of fluid impermeable material extends to the first end region, the second end region having a second width that is at least about 2 times greater than the first end region;
a fluid permeable material extending at least partially between the first end region and the second end region, at least some of the fluid permeable material positioned to be at least proximate to a urethra of a subject when the urine collection device is secured to the subject; and
a conduit having an inlet positioned at least proximate to reservoir of void space in the first region of fluid impermeable material, the conduit extending from the inlet between at least some of the fluid permeable material and the second region of fluid impermeable material and to the second end region of the urine collection device, wherein the conduit is configured to couple to a vacuum source effective to pull urine collected in the reservoir into the inlet and through the conduit for removal from the urine collection device.

10. The urine collection device of claim 9, wherein the at least some of the fluid permeable material positioned to be at least proximate to the urethra of the subject forms a substantially concave surface on the urine collection device.

11. The urine collection device of claim 10, wherein the at least some of the at the fluid permeable material includes a wicking material forming the substantially concave surface positioned to be at least proximate to the urethra of the subject.

12. The urine collection device of claim 9, wherein the fluid permeable material includes at least a wicking material configured to direct the urine towards the reservoir and the inlet of the conduit.

13. The urine collection device of claim 12, wherein the second region of fluid impermeable material includes a fluid impermeable film.

14. The urine collection device of claim 13, wherein the wicking material forms at least a portion of a front side of the urine collection device and the fluid impermeable film forms of the second region of fluid impermeable material forms a rear side of the urine collection device.

15. The urine collection device of claim 9, wherein the fluid impermeable layer defines an aperture at the second end region and the conduit extends through the aperture.

16. A urine collection device, comprising:
a fluid impermeable layer;
a fluid permeable material secured to the fluid impermeable layer, at least some of the fluid permeable material positioned to be at least proximate to a urethra of a subject when the urine collection device is secured to the subject;
a first member extending longitudinally between the fluid permeable material and the fluid impermeable layer;
a second member extending longitudinally between the fluid permeable material and the impermeable layer; and
a connector secured to the first member and the second member, the first member and the second member forming a V-shaped configuration in the urine collection device when secured to the connector;
wherein at least one member of the first member or the second member is substantially tubular and in fluid communication with the connector and configured to transport urine from within the urine collection device when fluidly coupled to a vacuum source for removal from the urine collection device.

17. The urine collection device of claim 16, wherein the at least some of the fluid permeable material positioned to be at least proximate to the urethra of the subject forms a substantially concave surface on the urine collection device.

18. The urine collection device of claim 17, wherein the at least some of the at the fluid permeable material includes a wicking material forming the substantially concave surface positioned to be at least proximate to the urethra of the subject.

19. The urine collection device of claim 16, wherein the fluid impermeable layer includes a fluid impermeable film.

20. The urine collection device of claim 19, wherein the wicking material forms at least a portion of a front side of the urine collection device and the fluid impermeable film forms of the second region of fluid impermeable material forms a rear side of the urine collection device.
